(19) United States
(12) Patent Application Publication
(10) Pub. No.: US 2022/0202615 A1
(43) Pub. Date: Jun. 30, 2022

(54) SYSTEMS AND METHODS FOR ABLATING OPHTHALMIC TISSUE

(71) Applicant: Alcon Inc., Fribourg (CH)

(72) Inventors: Zsolt Bor, San Clemente, CA (US); Mario Klafke, Mainaschaff (DE); Johannes Krause, Nürnberg (DE); Keith Watanabe, Irvine, CA (US)

(21) Appl. No.: 17/644,810

(22) Filed: Dec. 17, 2021

Related U.S. Application Data
(60) Provisional application No. 63/130,518, filed on Dec. 24, 2020.

(51) Int. Cl. A61F 9/008

(52) U.S. Cl. CPC A61F 9/00814; A61B 2017/00181; A61F 2009/00897; A61F 9/00804

(57) ABSTRACT

In certain embodiments, an ophthalmic surgical system for ablating tissue of an eye comprises controllable components (such as a light source and a scanner), optical elements, and a computer. The light source generates a light beam comprising pulses, where a propagation direction of the light beam defines a z-axis. The scanner directs a focal point of the light beam in an xy-plane orthogonal to the z-axis. The optical elements shape and focus the focal point of the light beam at a treatment region of the eye. The computer instructs one or more of the controllable components to generate the light beam comprising the pulses, where each pulse has a fluence greater than 1 J/cm². An optical element of the optical elements focuses the focal point of the light beam with a spot size of less than 0.4 mm at the treatment region according to a focal spot pattern.

### TECHNICAL FIELD

The present disclosure relates generally to ophthalmic surgical systems and methods, and more particularly to ablation systems and methods for ablating ophthalmic tissue.

### BACKGROUND

Laser ablation removes material from a surface by irradiating it with a laser beam. In ophthalmic surgery, an ablation procedure typically uses an excimer laser to reshape the cornea to change its refractive properties. During the procedure, the excimer laser beam is directed towards the cornea according to a laser focal spot pattern. The beam forces the molecules to detach from each other, and material is removed to yield a desired corneal shape.

The depth accuracy of the laser beam is important to achieve desired refractive results. In addition, the laser beam heats the tissue, so the temperature of the tissue should be controlled to avoid tissue damage. In certain embodiments, the heat in the cornea diffuses into the deeper laying stromal layers to about 100 um. The heat may diffuse to the anterior chamber of the eye, which may cause unwanted side effects. Certain known systems fail provide satisfactory accuracy and temperature control.

### BRIEF SUMMARY

In certain embodiments, an ophthalmic surgical system for ablating tissue of an eye comprises controllable components, optical elements, and a computer. The controllable components comprise a light source and a scanner. The light source generates a light beam comprising pulses, where a propagation direction of the light beam defines a z-axis. The scanner directs a focal point of the light beam in an xy-plane orthogonal to the z-axis. The optical elements shape and focus the focal point of the light beam at a treatment region of the eye. The computer instructs one or more of the controllable components to generate the light beam comprising the pulses, where each pulse has a fluence greater than 1 joule per square centimeter (J/cm²). An optical element of the optical elements focuses the focal point of the light beam with a spot size of less than 0.4 millimeters (mm) at the treatment region of the eye according to a focal spot pattern.

Embodiments may include none, one, some, or all of the following features:

The optical elements comprise a beam shaper configured to shape the light beam. The beam shaper may be an aperture.

The optical elements comprise a beam homogenizer. The beam homogenizer may be a diffractive element.

The optical element of the plurality of optical elements comprises an objective configured to focus the light beam.

The system yields a stabilization factor of less than 0.35, where the stabilization factor describes a percentage of change of an ablation depth of a pulse caused by a 1% change of the fluence of the pulse.

The pulses yields an ablation depth greater than 0.760 um, such as greater than 0.9 um.

Each pulse has a fluence greater than 1.2 joule per square centimeter (J/cm²).

In certain embodiments, a method for ablating tissue of an eye, comprises: generating, by a light source of a plurality of controllable components, a light beam comprising a plurality of pulses, a propagation direction of the light beam defining a z-axis; directing, by a light source of the plurality of controllable components, a focal point of the light beam in an xy-plane orthogonal to the z-axis; shaping and focusing, by a plurality of optical elements, the focal point of the light beam at a treatment region of the eye; instructing, by a computer, one or more of the controllable components to generate the light beam comprising the plurality of pulses, each pulse having a fluence greater than 1 joule per square centimeter (J/cm²); and focusing, by an optical element of the plurality of optical elements, the focal point of the light beam with a spot size of less than 0.4 millimeters (mm) at the treatment region of the eye according to a focal spot pattern.

Embodiments may include none, one, some, or all of the following features:

The shaping the focal point comprises shaping, by a beam shaper, the light beam. The beam shaper may be an aperture.

The shaping the focal point comprises homogenizing, by a beam homogenizer, the light beam. The beam homogenizer may be a diffractive element.

The optical element of the plurality of optical elements comprises an objective configured to focus the light beam.

The pulses yield a stabilization factor of less than 0.35, where the stabilization factor describes a percentage of change of an ablation depth of a pulse caused by a 1% change of the fluence of the pulse.

The pulses yield an ablation depth greater than 0.760 um, such as greater than 0.9 um.

In certain embodiments, an ophthalmic surgical system for ablating tissue of an eye comprises controllable components, optical elements, and a computer. The controllable components comprise a light source and a scanner. The optical elements comprises: a beam shaper configured to shape the light beam, the beam shaper comprising an aperture; and a beam homogenizer configured to homogenize the light beam, the beam homogenizer comprising a diffractive element. The light source generates a light beam comprising pulses, where a propagation direction of the light beam defines a z-axis. The scanner directs a focal point of the light beam in an xy-plane orthogonal to the z-axis. The optical elements shape and focus the focal point of the light beam at a treatment region of the eye. The computer instructs one or more of the controllable components to generate the light beam comprising the pulses, where each pulse has a fluence greater than 1 joule per square centimeter (J/cm²) and the pulses yield an ablation depth greater than 0.9 um. An optical element comprising an objective focuses the focal point of the light beam with a spot size of less than 0.4 millimeters (mm) at the treatment region of the eye according to a focal spot pattern. The system yields a stabilization factor of less than 0.35, where the stabilization factor describes a percentage of change of an ablation depth of a pulse caused by a 1% change of the fluence of the pulse.

### BRIEF DESCRIPTION OF THE DRAWINGS

FIG. 1 illustrates an example of an ophthalmic ablation system that ablates the corneal tissue of an eye to treat presbyopia, according to certain embodiments;

FIG. 2 illustrates examples of a light source, a scanner, one or more optical elements, and a focusing objective that may be used in the system of FIG. 1;

FIG. 3 illustrates a graph that describes how the laser pulse fluence changes as pulses penetrate corneal tissue;

FIG. 4 illustrates a graph that describes ablation depths of pulses of different laser pulse fluences;

FIG. 5 illustrates a graph that describes how higher incident fluences can yield improved depth accuracy;

FIGS. 6A and 6B illustrate graphs that describe how increased fluence does not increase the temperature, contrary to expectation;

FIG. 7 illustrates how smaller spots created by laser pulses cool more efficiently than larger spots; and

FIG. 8 illustrates a method for ablating a cornea of an eye that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In certain embodiments, an ophthalmic surgical system generates a light beam with a plurality of pulses, where each pulse has a fluence greater than 1 joule per square centimeter (J/cm₂). The system focuses the focal point of the light beam with a spot size of less than 0.4 millimeters (mm) at the eye. Contrary to the expectation that increasing the fluence increases the temperature of the tissue, the increased fluence does not increase the temperature. In addition, the increased fluence can provide improved depth accuracy.

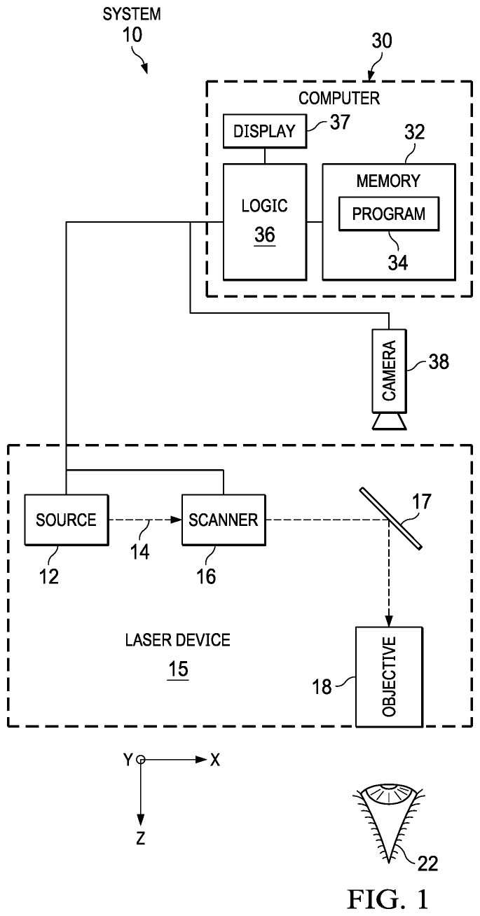

FIG. 1 illustrates an example of an ophthalmic ablation system 10 that ablates the corneal tissue of eye 22 to treat presbyopia, according to certain embodiments. System 10 may be used for different types of procedures. For example, laser in-situ keratomileusis (LASIK) involves cutting a flap in the cornea and then using system 10 to ablate the cornea. As another example, in photo refractive keratectomy (PRK), the epithelium is removed, e.g., chemically or mechanically, and then system 10 is used to ablate the cornea.

In the illustrated example, system 10 includes a laser device 15, a camera 38, and a control computer 30, coupled as shown. Laser device 15 includes controllable components, such as a light source (e.g., a laser source 12), a scanner 16, one or more optical elements 17, and/or a focusing objective 18, coupled as shown. Computer 30 includes logic 36, a memory 32 (which stores a computer program 34), and a display 37, coupled as shown. For ease of explanation, the following xyz-coordinate system is used: The z-direction is defined by the propagation direction of the laser beam, and the xy-plane is orthogonal to the propagation direction. Other suitable xyz-coordinate systems may be used.

Turning to the parts of system 10, a light source generates a light beam that ablates tissue of eye 22 according to a focal spot pattern. The light beam may have a wavelength of, e.g., less than 300 nm. In the illustrated example, the light source is a laser source 12 that generates a laser beam that ablates tissue of eye 22 according to a laser focal spot pattern. Laser source 12 may be an excimer, solid-state, or other suitable laser.

A focal spot pattern may define x and y (and perhaps z) coordinates for positions at which laser radiation pulses are to be directed in the treatment region (e.g., exposed surface of eye). The focal spot pattern may be determined from an ablation profile, which indicates the volume of tissue to be removed at particular x, y positions of the cornea. Given the volume of tissue ablated per pulse, the number of pulses to be directed at an x, y position can be calculated from the volume of tissue defined by the ablation profile.

Scanner 16 laterally and/or longitudinally directs the focal point of the laser beam. The lateral direction refers to directions orthogonal to the direction of beam propagation, i.e., the x, y directions. Scanner 16 may laterally direct the laser beam in any suitable manner. For example, scanner 16 may include a pair of galvanometrically-actuated scanner mirrors that can be tilted about mutually perpendicular axes. As another example, scanner 16 may include an electro-optical crystal that can electro-optically steer the laser beam.

The longitudinal direction refers to the direction parallel to the laser beam propagation, i.e., the z-direction. Scanner 16 may longitudinally direct the laser beam in any suitable manner. For example, scanner 16 may include a longitudinally adjustable lens, a lens of variable refractive power, or a deformable mirror that can control the z-position of the beam focus. The components of scanner 16 may be arranged in any suitable manner along the beam path, e.g., in the same or different modular units.

One (or more) optical elements 17 direct the laser beam towards focusing objective 18. An optical element 17 can act on (e.g., transmit, reflect, refract, diffract, collimate, condition, shape, focus, modulate, and/or otherwise act on) a laser beam. Examples of optical elements include a lens, prism, mirror, diffractive optical element (DOE), holographic optical element (HOE), and spatial light modulator (SLM). In the example, optical element 17 is a mirror. Focusing objective 18 focuses the focal point of laser beam towards a point of eye 22. In the example, focusing objective 18 is an objective lens, e.g., an f-theta objective.

Camera 38 records images of the eye 22. Examples of camera 38 include a video, an optical coherence tomography, or an eye-tracking camera. Camera 38 delivers image data, which represent recorded images of the eye 22, to computer 30. Computer 30 may carry out image processing on the image data to monitor ablation of eye 22.

Computer 30 controls components of system 10 in accordance with computer program 34. For example, computer 30 controls components (e.g., laser source 12, scanner 16, optical elements 17, and/or focusing objective 18) to focus the laser beam of laser device 15 at eye 22 and to ablate at least a portion of eye 22 according to an ablation profile.

In certain embodiments, computer 30 instructs laser device 15 to generate a laser beam with a plurality of pulses, where each pulse has a fluence greater than 1 joule per square centimeter (J/cm²), such as a value in the range of 1.0 to 1.2, 1.2 to 1.4, 1.4 to 1.6. 1.6 to 2.0, and/or greater than 2.0 J/cm². The pulses may have any suitable local repetition rate, e.g., 30 to 100 pulses per second. The focal point of the laser beam is focused with a spot size of less than 0.4 millimeters (mm) at the treatment region of eye, such as a value in the range of 0.4 to 0.3, 0.3 to 0.2, and/or less than 0.2 mm. The spot size may be described in any suitable manner, e.g., as 1/e2 beam radius for a Gaussian beam and Full Width at Half Maximum (FWHM) for a non-Gaussian beam. Contrary to the expectation that increasing fluence increases the temperature of the tissue, the increased fluence does not increase the temperature. In addition, the increased fluence can provide improved accuracy.

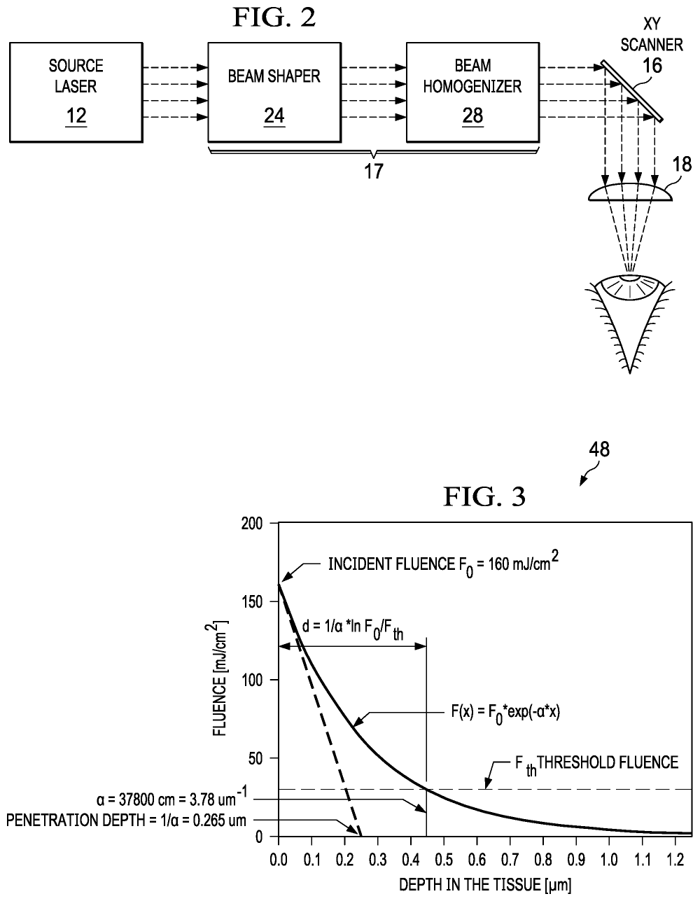

FIG. 2 illustrates examples of laser source 12, scanner 16, one or more optical elements 17, and focusing objective 18 that may be used in system 10 of FIG. 1. In the illustrated example, optical elements 17 includes a beam shaper 24 and a beam homogenizer 28. Beam shaper 24 is an optical element (e.g., an aperture) that changes the cross-sectional shape of a laser beam, e.g., from rectangular to circular. Beam homogenizer 26 is an optical element (e.g., a diffractive element) that smooths out the irregularities in a laser beam profile to create a more homogeneous one. Beam shaper 24 and beam homogenizer 26 may have any suitable arrangement. For example, a laser beam may pass through beam homogenizer 26 and then beam shaper 24, or vice-versa.

FIG. 3 illustrates a graph 48 that describes how the laser pulse fluence changes as pulses penetrate corneal tissue. In general, pulses with a higher incidence fluence ablate more tissue. Function F(x) describes laser fluence relative to depth x of the cornea:

$$F ( x ) = {{F}_{0}}^{*} exp ( - {a}^{*} x ) \qquad ( 1 )$$

where F₀ is the incident fluence, and a is the absorption coefficient of the tissue.

Tissue that is exposed to a fluence higher than an ablation threshold Fₜₕ is ablated away. The ablation threshold Fₜₕ for corneal tissue is approximately 30 mJ/cm². The ablation depth can be calculated from Equation (1) as:

$$d = 1 / {a}^{*} ln ( {F}_{0} / {F}_{th} ) \qquad ( 2 )$$

In the example, graph 48 describes a 193 nm excimer laser, where a=3.78 micrometers⁻¹ (um⁻¹), and the penetration depth 1/a=0.265 micrometers (um).

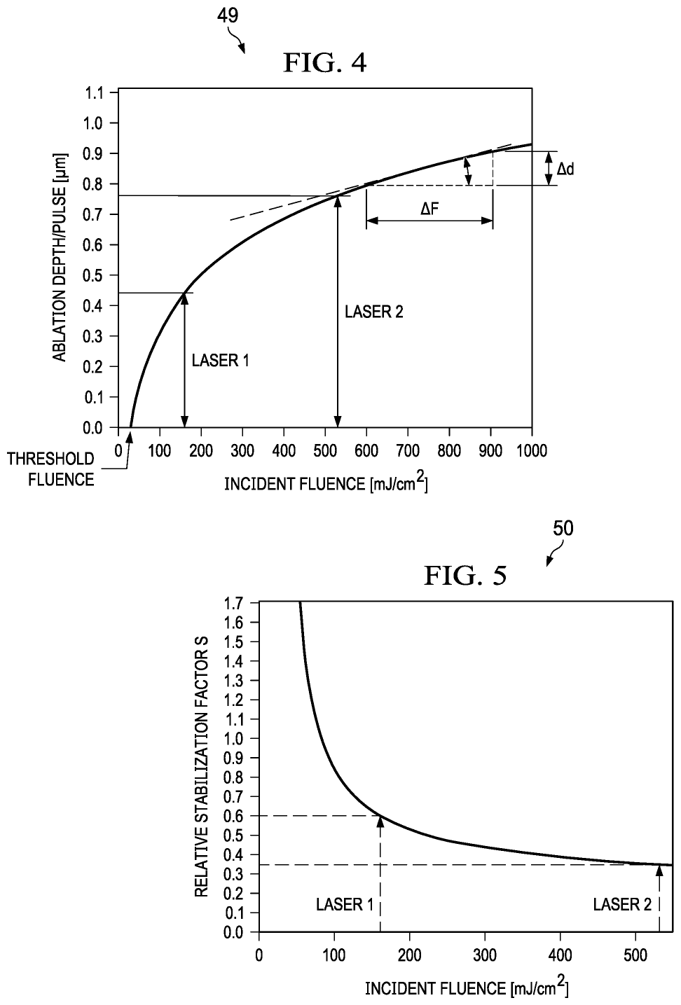

FIG. 4 illustrates a graph 49 that describes ablation depths of pulses of different laser pulse fluences. In the example, a=3.78 micrometers⁻¹ (um⁻¹), and Fₜₕ=30 mJ/cm². Laser 1 generates pulses with incident fluence F₀1=160 mJ/cm², and Laser 2 generates pulses with incident fluence F₀2=530 mJ/cm². According to Equation (2), Laser 1 has ablation depth d1=0.443 um, and Laser 2 has ablation depth d2=0.760 um. Accordingly, Laser 2 removes 0.76/0.443=1.72 times more tissue per pulse. The same holds for pulses with fluences greater than 1 J/cm². For example, pulses of system 10 may yield ablation depths greater than 0.760 um, e.g., greater than 0.8 um or 0.9 um.

FIG. 5 illustrates a graph 50 that describes how higher incident fluences can yield improved depth accuracy. In practice, the ablation depth is affected by uncontrolled variation of the incident fluence. Uncontrolled variation may be caused by, e.g., shot-to-shot variations of the laser pulse energy; long term drift of laser energy; absorption of incident laser energy by the ablation plume of the previous pulse; and variation of the laser spot profile.

Stabilization factor S describes the percentage of change of the ablation depth caused by a 1% change of the incident fluence:

$$S = ( Δ d / d ) / ( Δ F / F ) \qquad ( 3 )$$

where d represents depth and F represents fluence. (See also FIG. 4 for Δd and ΔF.) A lower stabilization factor S indicates stronger stabilization, and a higher stabilization factor S indicates weaker stabilization. Stabilization factor S can be calculated from Equation (2) as:

$$S = 1 / ( ln ( {F}_{0} / {F}_{th} ) \qquad ( 4 )$$

Note that the ratio of stabilization factors is also the ratio of ablation depths d1/d2:

$$( d 2 / d 1 ) = ( S 1 / S 2 ) = ln ( {F}_{0} 2 / {F}_{th} ) / ln ( {F}_{0} 1 / {F}_{th} ) = 1.72 \qquad ( 5 )$$

Graph 50 shows the stabilization factors for Laser 1 with incident fluence F₀1=160 mJ/cm² and Laser 2 with incident fluence F₀2=530 mJ/cm². According to graph 50, the stabilization factor for Laser 1 is S1=0.6, and the stabilization factor for Laser 2 is S2=0.348. That is, Laser 2 with higher incident fluence has 0.6/0.348=1.72 times stronger stabilization. In certain embodiments, system 10 may yield a stabilization factor of less than 0.35, e.g., less than 0.30. Accordingly, higher incident fluences can yield improved depth accuracy.

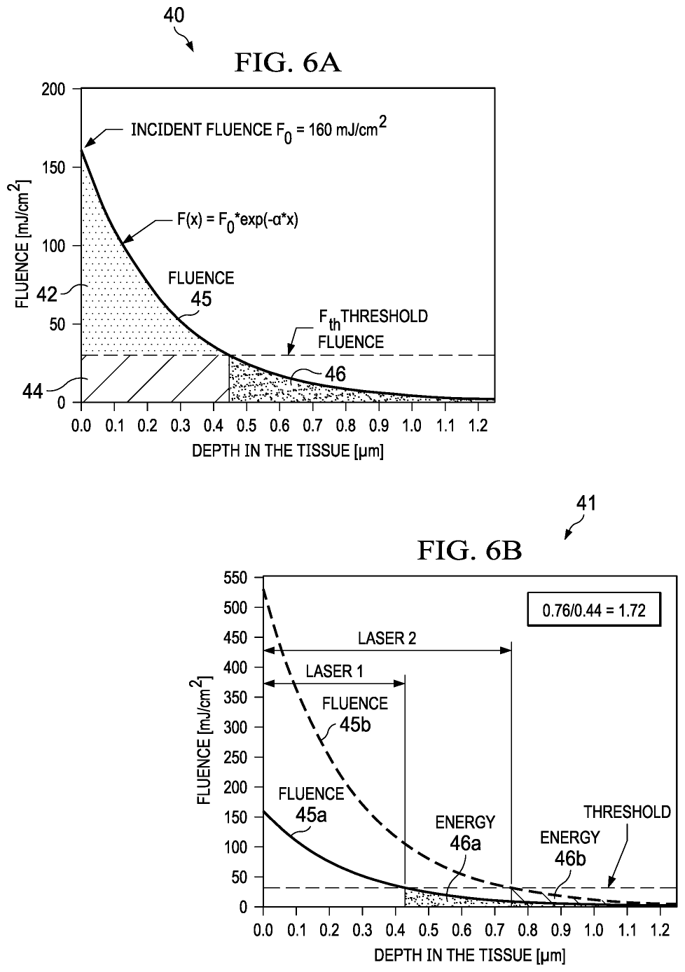

FIGS. 6A and 6B illustrate graphs 40, 41, respectively, that describe how increased fluence does not increase the temperature, contrary to expectation. FIG. 6A illustrates graph 40, which shows the laser pulse fluence F(x) 45 relative to depth x of corneal tissue. The incident pulse fluence F₀ is the fluence F(x), where x=0. In the example, the incident fluence F₀=160 mJ/cm². Graph 40 also shows the energies 42, 44, 46 of the fluence F(x) 45. Energy 42 represents the energy needed to heat the cornea from body temperature to 100° C. and to convert corneal water to vapor. Energy 44 represents energy converted to kinetic energy that ejects the ablation plume and ablation debris from the surface. Energy 46 represents the energy remaining in the tissue that is converted to heat.

FIG. 6B illustrates graph 41, which describes how the heat remaining in the cornea (represented by energy 46) is independent of the incident pulse fluence F₀. Graph 41 shows fluence F(x) 45a and energy 46a for Laser 1 with incident fluence F₀1=160 mJ/cm², and fluence F(x) 45b and energy 46b for Laser 2 with incident fluence F₀2=530 mJ/cm². As can be calculated from the incident fluences F₀ of Lasers 1 and 2 and Equation (2), Laser 2 ablates 1.72 times more tissue per pulse than Laser 1. Accordingly, Laser 2 uses 1.72 times fewer pulses to ablate to the same ablation depth. Moreover, the value of 1.72 can be increased by increasing the fluence of Laser 2 relative to that of Laser 1. The same holds for pulses with fluences greater than 1 J/cm².

In the example, energy 46a, representing the heat remaining in the tissue per pulse, for Laser 1 is the same as energy 46b of Laser 2. That is, a 160 mJ/cm² pulse and a 530 mJ/cm² pulse leave the same amount of heat in the cornea. Higher fluence pulses ablate more from the cornea and increase the kinetic energy of the plume and debris (represented by energy 44), but leave the same amount of the heat in the cornea (represented by energy 46).

As shown above, Laser 2 uses 1.72 times fewer pulses than Laser 1 to ablate to the same ablation depth. Moreover, a 160 mJ/cm² pulse and a 530 mJ/cm² pulse leave the same amount of heat in the cornea. Therefore, Laser 2 deposits 1.72 times less heat into the tissue. The same holds for pulses with fluences greater than 1 J/cm². Thus, increased fluence does not increase the temperature, contrary to expectation.

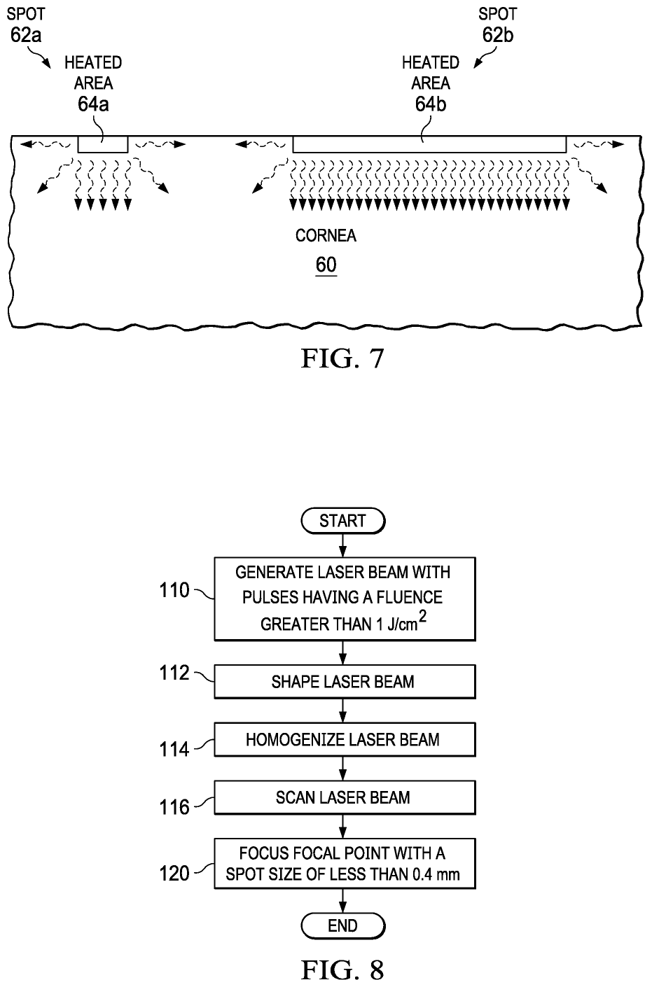

FIG. 7 illustrates how smaller spots created by laser pulses cool more efficiently than larger spots. Spots 62 (62a, 62b) of a cornea 60 are heated by laser pulses. Spot 62a is smaller than spot 62b, and yields a smaller heated area 64a than the heated area 64b of larger spot 62b. Three-dimensional (3D) heat dispersion, or cooling, occurs at the edges of heated areas 64 (64a, 64b), and one-dimensional (1D) heat dispersion occurs at the inner portions of heated areas 64 (64a, 42b). 3D heat dispersion is more effective than the 1D heat dispersion.

A greater percentage of the heat of smaller spot 62a is dispersed via 3D heat dispersion than the percentage of the heat of larger spot 62b dispersed via 3D dispersion. As a result, smaller spot 62a cools more efficiently than larger spot 62b. Accordingly, system 10 focuses the laser beam with a spot size of less than 0.4 mm.

FIG. 8 illustrates a method for ablating a cornea of an eye that may be performed by system 10 of FIG. 1, according to certain embodiments. In the embodiments, computer 30 instructs controllable components of system 10 to perform certain steps of the method according to an ablation profile.

The method starts at step 110, where laser source 12 generates a laser beam with a plurality of pulses. Each pulse has a fluence greater than 1 joule per square centimeter (J/cm²). Beam shaper 24 shapes the laser beam at step 112, and beam homogenizer 28 homogenizes the laser beam at step 114. In other embodiments, beam homogenizer 28 homogenizes the laser beam at step 112, and beam shaper 24 shapes the laser beam at step 114. Scanner 16 scans the laser beam at step 116. Scanner may scan the laser beam according to a laser focal spot pattern corresponding to the ablation profile. Objective 18 focuses the laser beam with a spot size of less than 0.4 mm at the eye at step 120. The method then ends.

A component (such as the control computer 30) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface (e.g., a Graphical User Interface (GUI)) is a type of interface that a user can utilize to interact with a computer. Examples of user interfaces include a display, touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by the electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic surgical system for ablating tissue of an eye, comprising:
a plurality of controllable components comprising:
a light source configured to generate a light beam comprising a plurality of pulses, a propagation direction of the light beam defining a z-axis;
a scanner configured to direct a focal point of the light beam in an xy-plane orthogonal to the z-axis; and
a plurality of optical elements configured to shape and focus the focal point of the light beam at a treatment region of the eye;
a computer configured to instruct one or more of the controllable components to:
generate the light beam comprising the plurality of pulses, each pulse having a fluence greater than 1 joule per square centimeter (J/cm²); and
an optical element of the plurality of optical elements configured to focus the focal point of the light beam with a spot size of less than 0.4 millimeters (mm) at the treatment region of the eye according to a focal spot pattern.

2. The ophthalmic surgical system of claim 1, the optical elements comprising:
a beam shaper configured to shape the light beam.

3. The ophthalmic surgical system of claim 2, the beam shaper comprising an aperture.

4. The ophthalmic surgical system of claim 1, the optical elements comprising:
a beam homogenizer configured to homogenize the light beam.

5. The ophthalmic surgical system of claim 4, the beam homogenizer comprising a diffractive element.

6. The ophthalmic surgical system of claim 1, the optical element of the plurality of optical elements comprising:
an objective configured to focus the light beam.

7. The ophthalmic surgical system of claim 1, the system yielding a stabilization factor of less than 0.35, the stabilization factor describing a percentage of change of an ablation depth of a pulse caused by a 1% change of the fluence of the pulse.

8. The ophthalmic surgical system of claim 1, the pulses yielding an ablation depth greater than 0.760 um.

9. The ophthalmic surgical system of claim 8, the pulses yielding an ablation depth greater than 0.9 um.

10. The ophthalmic surgical system of claim 1, each pulse having a fluence greater than 1.2 joule per square centimeter (J/cm²).

11. A method for ablating tissue of an eye, comprising:
generating, by a light source of a plurality of controllable components, a light beam comprising a plurality of pulses, a propagation direction of the light beam defining a z-axis;
directing, by a light source of the plurality of controllable components, a focal point of the light beam in an xy-plane orthogonal to the z-axis;
shaping and focusing, by a plurality of optical elements, the focal point of the light beam at a treatment region of the eye;
instructing, by a computer, one or more of the controllable components to generate the light beam comprising the plurality of pulses, each pulse having a fluence greater than 1 joule per square centimeter (J/cm²); and
focusing, by an optical element of the plurality of optical elements, the focal point of the light beam with a spot size of less than 0.4 millimeters (mm) at the treatment region of the eye according to a focal spot pattern.

12. The method of claim 11, the shaping the focal point comprising:
shaping, by a beam shaper, the light beam.

13. The method of claim 12, the beam shaper comprising an aperture.

14. The method of claim 11, the shaping the focal point comprising:
homogenizing, by a beam homogenizer, the light beam.

15. The method of claim 14, the beam homogenizer comprising a diffractive element.

16. The method of claim 11, the optical element of the plurality of optical elements comprising an objective configured to focus the light beam.

17. The method of claim 11, the pulses yielding a stabilization factor of less than 0.35, the stabilization factor describing a percentage of change of an ablation depth of a pulse caused by a 1% change of the fluence of the pulse.

18. The method of claim 11, the pulses yielding an ablation depth greater than 0.760 um.

19. The method of claim 18, the pulses yielding an ablation depth greater than 0.9 um.

20. An ophthalmic surgical system for ablating tissue of an eye, comprising:
a plurality of controllable components comprising:
a light source configured to generate a light beam comprising a plurality of pulses, a propagation direction of the light beam defining a z-axis;
a scanner configured to direct a focal point of the light beam in an xy-plane orthogonal to the z-axis; and
a plurality of optical elements configured to shape and focus the focal point of the light beam at a treatment region of the eye, the optical elements comprising:
a beam shaper configured to shape the light beam, the beam shaper comprising an aperture; and
a beam homogenizer configured to homogenize the light beam, the beam homogenizer comprising a diffractive element;
a computer configured to instruct one or more of the controllable components to:
generate the light beam comprising the plurality of pulses, each pulse having a fluence greater than 1 joule per square centimeter (J/cm²), the pulses yielding an ablation depth greater than 0.9 um; and
an optical element of the plurality of optical elements configured to focus the focal point of the light beam with a spot size of less than 0.4 millimeters (mm) at the treatment region of the eye according to a focal spot pattern, the optical element comprising an objective configured to focus the light beam, the system yielding a stabilization factor of less than 0.35, the stabilization factor describing a percentage of change of an ablation depth of a pulse caused by a 1% change of the fluence of the pulse.
